(19) United States
(12) Patent Application Publication
(10) Pub. No.: US 2024/0139447 A1
(43) Pub. Date: May 2, 2024

(54) CONTROL SYSTEM FOR A HUMIDIFICATION UNIT AND PROCESS FOR GENERATING A CONTROL OUTPUT

(71) Applicant: Drägerwerk AG & Co. KGaA, Lübeck (DE)

(72) Inventor: Tilman von BLUMENTHAL, Lübeck (DE)

(21) Appl. No.: 18/493,888

(22) Filed: Oct. 25, 2023

(30) Foreign Application Priority Data
Oct. 28, 2022 (DE) 10 2022 128 606.1

(51) Int. Cl. A61M 16/00; A61M 16/10; A61M 16/16; G01P 5/02

(52) U.S. Cl. CPC A61M 16/024; A61M 16/0003; A61M 16/109; A61M 16/16; G01P 5/02; A61M 2205/3327

(57) ABSTRACT

A control system is provided for a humidification unit configured to humidify a breathing gas stream such that a predetermined water content is achieved. The control system includes a thermal anemometer arranged downstream of the humidification unit, and further includes a processor unit. The processor unit is configured to receive a measurement signal from the thermal anemometer and determine therefrom a downstream fluid velocity, receive information about an expected fluid velocity corresponding to the predetermined water content, perform a comparison as to whether there is a discrepancy between the downstream fluid velocity and the expected fluid velocity, and generate a control output indicative of a comparison result. A process generates a control output using the control system.

### CROSS REFERENCE TO RELATED APPLICATIONS

This application claims the benefit of priority under 35 U.S.C. § 119 of German Application 10 2022 128 606.1, filed Oct. 28, 2022, the entire contents of which are incorporated herein by reference.

### TECHNICAL FIELD

The present invention relates to a control system for a humidification unit which is configured to humidify a breathing gas stream such that a predetermined water content is achieved. The invention further relates to a process of generating a control output using the control system.

### BACKGROUND

Humidification units, which are configured to humidify a breathing gas stream in such a way that a predetermined water content is achieved, are used in medical devices, such as in ventilators and in anesthesia machines. An example of such a humidification unit is an evaporator.

If humidification of the respiratory gas flow (breathing gas flow) does not take place to reach the predetermined water content but, for example, to reach a water content that is too low, this can cause damage to a ventilated patient. If the water content is increased above the predetermined water content, condensation may occur, for example, which is undesirable.

### SUMMARY

There is therefore a need to be able to reliably and easily check a humidification system for a predetermined water content. There is also the need for a quick check.

It is an object of the present invention to provide a control system for a humidification unit and a process for generating a control output with the control system, whereby humidification to the predetermined water content can be checked reliably, quickly and in a simple manner.

This object is attained by control system features according to this disclosure and by corresponding process features according to this disclosure.

Advantageous embodiments are disclosed herein.

According to the invention, a control system is provided for a humidification unit which is configured to humidify a breathing gas stream such that a predetermined water content is achieved.

The control system has a thermal anemometer located downstream of the humidification unit. The control system further comprises a processor unit. The processor unit is configured to: receive a measurement signal from the thermal anemometer and determine therefrom a downstream fluid velocity, receive information regarding an expected fluid velocity corresponding to the predetermined water content, perform a comparison as to whether there is a discrepancy between the downstream fluid velocity and the expected fluid velocity, and generate a control output indicative of a comparison result.

Within the scope of the invention, it was recognized that the downstream fluid velocity can be determined particularly advantageously with a thermal anemometer (hereinafter also referred to simply as “anemometer”), since this can also respond sufficiently quickly to dynamically changing measurement conditions due to its low thermal inertia, i.e., for example, with a step response time t90 of less than 20 ms.

By comparing the downstream fluid velocity, which can be determined using the anemometer, with the fluid velocity expected during humidification to the predetermined water content, it is possible to check whether or not the predetermined water content has been reached. Based on this comparison, a control output indicating the comparison result can then be generated.

The comparison between the downstream fluid velocity and the expected fluid velocity according to the invention thus allows a qualitative statement as to whether there is a deviation and consequently allows a check as to whether the humidification unit is functioning properly or not.

Since the particularly fast measuring behavior of the technically simple anemometer can be used for this purpose, the control output can be generated quickly and easily compared with a hygrometer, for example.

The control output indicating the comparison result (e.g. deviation exists or does not exist) can optionally be output or otherwise processed.

For example, the control output can be processed as part of a control or regulation of the humidification unit or of a medical device with which the humidification unit interacts, in order, for example, to influence a heating output and/or water dosage of the humidification unit accordingly.

According to the invention, the predetermined water content can be specified, for example, as absolute humidity. For example, the predetermined water content can also be specified as specific humidity or relative humidity.

According to the invention, a breathing gas flow is understood to mean a flow of a breathing gas, i.e., a gas that is breathable for humans, which may, for example, have breathing air as a component and may comprise further components, such as an additional oxygen component and/or an anesthetic. Also included are breathing gases comprising pure oxygen, as may be required for certain medical maneuvers.

According to the invention, a humidification unit can be any unit suitable for humidifying the breathing gas stream, such as an evaporator or nebulizer.

According to the invention, a processor unit can be any unit configured for data processing, for example an integrated circuit or an embedded system, in particular a microprocessor or a computer. The processing unit may be configured to receive and transmit information by wire or wirelessly. The processing unit may include a memory unit. In the context of the invention, receiving information includes both obtaining the information from a separate unit and reading information stored in a memory unit of the processor unit or otherwise, for example as predetermined information.

According to the invention, a process for generating a control output with a control system according to the invention is further provided.

Steps of this process are: receiving the downstream thermal anemometer measurement signal, determining the downstream fluid velocity from the measurement signal, receiving the expected fluid velocity information, performing the comparison of whether there is a discrepancy between the downstream fluid velocity and the expected fluid velocity, and generating the control output indicating the comparison result.

The process according to the invention also provides the effects and advantages described for the control system.

Preferably, the processing unit is configured to determine the downstream fluid velocity using calibration parameters corresponding to the predetermined water content.

In this way, it is possible to further simplify the control system. In this respect, the downstream fluid velocity can be determined particularly easily by merely making a determination based on calibration parameters.

For example, the downstream fluid velocity can be determined using the equation below, which is based on King's law:

$$M 2 = {[ \frac{[ \frac{{i}^{2} \cdot R}{( {T}_{w} - {T}_{f} )} - A ]}{B} ]}^{\frac{1}{2}} .$$

Here M2 denotes the downstream fluid velocity, i denotes an electric current with which the anemometer can be heated via an electric resistance R of the anemometer, Tw denotes a temperature of the anemometer, Tf denotes a temperature of the breathing gas stream, and A and B denote calibration parameters, the calibration parameters in this example being substance-dependent, i.e. dependent on the components present in the breathing gas. Components in this context refer to the constituents of the breathing gas and the water vapor present in it.

The parameter Tf can, for example, be measured by using a temperature sensor such as a thermometer in the breathing gas flow or can be predetermined. The parameter Tw can be kept constant by varying i, which may eliminate the need to determine Tw. Alternatively, i can be kept constant, whereby Tw can be determined indirectly by measuring R, for example.

Within the scope of the invention, it was recognized that the calibration parameters A and B are proportional to the specific humidity s, i.e., dependent on the amount of water vapor contained in the breathing gas and thus dependent on the water content of the breathing gas stream. Thus, for a breathing gas with specific humidity s, the following equations are obtained:

A=(1−s) a_{gas}+s·a_{H2O},

B=(1−s) b_{gas}+s·b_{H2O},

where a_{gas} and b_{gas} are gas-specific constants of the dry breathing gas and a_{H2O} and b_{H2O} are constants of the water content in the breathing gas, i.e. water-specific constants.

It was also recognized that for breathable gases the factors a_{H2O} and b_{H2O} are always larger than the factors a_{gas} and b_{gas}.

In order to determine the calibration parameters for a water content corresponding to the predetermined water content, a calibration may be performed with a stream of breathing gas that has been humidified to the predetermined water content. The calibration parameters thus obtained and/or the downstream fluid velocity corresponding thereto may, for example, be stored as predetermined information in a memory unit and provided to the processor unit or otherwise provided to the processor unit.

The determination of the downstream fluid velocity based on the calibration parameters is therefore incorrect for any water content that deviates from the predetermined water content. If, on the other hand, the water content corresponds to the predetermined water content, the determination provides an accurate value.

Thus, in the case of a deviation of the determined downstream fluid velocity from the expected fluid velocity, the comparison provides the qualitatively correct statement that a deviation exists, which, however, is not correct in terms of amount. In other words, in this preferred embodiment, there is a determination by estimation. However, this is sufficient for the generation of only a control output that indicates the comparison result, e.g., indicates whether a deviation is present or not.

Overall, a control output can thus be generated in a simple manner and consequently the control system can be provided in a simple manner.

As an alternative to the determination of the downstream fluid velocity using the equation based on King's law described above, thermodynamic heat balances can also be established at the anemometer and converted into differential equations. These can be solved by iterative procedures to find a solution for the humidity or downstream fluid velocity that is not only qualitatively but also quantitatively accurate. Alternatively to an iterative procedure, a linearization around an operating point of the anemometer can be performed. Further alternatively, instead of the continuous equation described above based on King's law, a plurality of equations can be used, each of these plurality of equations being optimized for a particular section of a flow region representing a working region of the thermal anemometer.

Preferably, the control system comprises a determination unit. In this case, the determination unit is configured to receive information about an upstream fluid velocity upstream from the humidification unit, to receive information about an upstream water content of the breathing gas stream, and to receive information about the predetermined water content.

The receiving steps comprise both the mere obtaining of the aforementioned information from a separate unit and the reading of the aforementioned information as predetermined information from a memory, for example a memory of the determination unit or the processor unit. Preferably, receiving also comprises determining the aforementioned information from predetermined information and/or from received measurement data or signals corresponding to the aforementioned information.

The determination unit is further adapted to determine the expected fluid velocity information from the predetermined water content, the upstream water content and the upstream fluid velocity and to provide, in particular send, the expected fluid velocity information to the processing unit.

By including the predetermined water content, the upstream water content, and the upstream fluid velocity in the determination of the expected fluid velocity, information about the expected fluid velocity can be generated that is independent of the actual humidification currently occurring in the humidification unit.

The control system can thus generate a control output that does not require any information from the humidification unit, whereby the control system provides particularly reliable operation even in cases where there is an error in the provision of information by the humidification unit.

The expected fluid velocity can be determined, for example, using the formula below:

$$M 3 = M 4 \cdot ( 1 + \frac{R \cdot T}{p} \cdot \frac{{f}_{soll} - {f}_{in}}{{M}_{w}} ) .$$

Where M3 denotes the expected fluid velocity, M4 denotes the upstream fluid velocity, fₛₒₗₗ denotes the predetermined water content relative to dry air, fᵢₙ denotes the upstream water content relative to dry air, M_{W} denotes the molar mass of water, R denotes the universal gas constant, T denotes the temperature of the breathing gas, and p denotes the pressure of the breathing gas stream.

For example, the predetermined water content fₛₒₗₗ may be 35.0 mg/l, the upstream water content fᵢₙ may be 0, for example when the humidification unit is supplied with breathing gas from a medical gas supply system, the molar mass of water is about 18.02 g/mol, the universal gas constant is about 8.31446 J/mol K, the temperature T of the breathing gas may be, for example, the body temperature of a human and may be, for example, 310.15 K, and the pressure p may be, for example, 101325 Pa. If the upstream fluid velocity M4 is 10.0 l/min in one example, the expected fluid velocity M3 is about 10.57 l/min, i.e., the expected increase in fluid velocity due to humidification corresponds to about 0.57 l/min in this example.

The temperature and/or the pressure and/or the upstream fluid velocity can be present as additional information, for example as signals from corresponding sensors, for example from a temperature sensor and/or a pressure sensor and/or a flow velocity sensor, and/or be a predetermined temperature and/or a predetermined pressure and/or a predetermined upstream fluid velocity, and in particular be present as predetermined information in the determination unit or in the processor unit.

As an alternative to determining the expected fluid velocity using the above preferred embodiment without taking into account any information from the humidification unit, for example, an amount of water currently added to the breathing gas stream by the humidification unit may be provided or information corresponding to this parameter may be provided as a signal for the determination unit or for the processor unit.

This can simplify the structure of the control system.

Such a signal can be provided, for example, by a pump that delivers water to the humidification unit or by a sensor that measures the amount of water currently added to the breathing gas stream.

Preferably, the determination unit is part of the processor unit.

Preferably, the control system has another thermal anemometer located upstream of the humidification unit to determine the upstream fluid velocity upstream of the humidification unit.

As described above, thermal anemometers are particularly suitable for reacting quickly in dynamic flow conditions of the breathing gas stream. By using a further thermal anemometer upstream, reliable and quickly available information about the upstream breathing gas flow can thus be obtained, which can particularly preferably be provided to the determination unit, if present.

Preferably, the thermal anemometer and/or the further thermal anemometer is a hot-wire anemometer.

This can further increase the response speed of the thermal anemometer, since a hot-wire anemometer has a lower thermal inertia than, for example, a film anemometer. Furthermore, the evaluation of the measurement signals of a hot-wire anemometer is particularly simple.

Preferably, the thermal anemometer and/or the further thermal anemometer is configured to be operated at a constant temperature.

Operation at a constant temperature refers both to a temperature that is constant relative to absolute zero and, alternatively, to a temperature that is constant relative to the (possibly variable) fluid temperature (also referred to as “excess temperature”). In the case of the control engineering setting of a constant overtemperature, undesirable temperature-dependent effects can be compensated.

By operating at a constant temperature, an improvement in the measurement behavior can thus be made possible.

Alternatively, the anemometer can be operated at a constant current, which can simplify the control and thus the control system as a whole.

Preferably, the thermal anemometer and/or the further thermal anemometer is formed as part of a Wheatstone bridge.

With such a configuration, the temperature dependency of the electrical resistance of the anemometer can be compensated in a simple way.

The thermal anemometer and/or the further thermal anemometer can also be connected to a digital voltmeter with operational amplifier to improve the measurement resolution.

Particularly preferably, in a combined embodiment, the anemometer is operated at a constant temperature by forming the anemometer as part of a Wheatstone's bridge. The bridge centers can then be connected, for example, to a differential amplifier whose output is connected in a feedback structure to the bridge tip.

The above-mentioned advantages, preferred features and effects result analogously for the process according to the invention.

In particular, in a preferred embodiment, the downstream fluid velocity is determined based on the calibration parameters corresponding to the predetermined water content.

Further, in a preferred embodiment, the process further comprises the steps of: receiving the upstream fluid velocity information, receiving the upstream water content information of the breathing gas stream, receiving the predetermined water content information, determining the expected fluid velocity information from the upstream water content, the predetermined water content, and the upstream fluid velocity, and providing the expected fluid velocity information.

These and other features and advantages of the present invention are also apparent from the following description of figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of an embodiment of the control system according to the invention;

FIG. 2 is a schematic view an embodiment of the control system according to the invention;

FIG. 3 is a flowchart showing an embodiment of the process according to the invention; and

FIG. 4 is a flowchart showing an embodiment of the process according to the invention.

### DESCRIPTION OF PREFERRED EMBODIMENTS

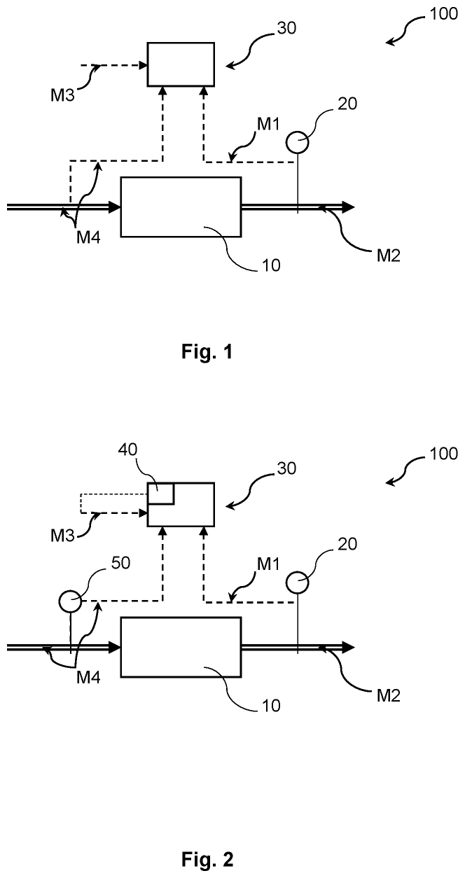

Referring to the drawings, FIGS. 1 and 2 show embodiments of the control system 100 according to the invention.

The control system 100 according to the invention is suitable for a humidification unit 10, for example a nebulizer, which is configured to humidify a breathing gas stream in such a way that a predetermined water content fₛₒₗₗ is achieved.

As shown in FIGS. 1 and 2, the control system 100 includes a thermal anemometer 20 located downstream of the humidification unit 10.

As shown in FIGS. 1, 2, the control system 100 further comprises a processor unit 30. In the illustrated embodiment, the processor unit 30 may be an integrated circuit or embedded system, in particular a microprocessor or a computer.

The processor unit 30 is configured to receive a measurement signal M1 from the thermal anemometer 20 and to determine a downstream fluid velocity M2 therefrom. The measurement signal M1 can be received in any way, for example by cable or wirelessly.

The processor unit 30 is further adapted to receive information about an expected fluid velocity M3 corresponding to the predetermined water content fₛₒₗₗ, and to perform a comparison as to whether there is a discrepancy between the downstream fluid velocity M2 and the expected fluid velocity M3.

In the embodiment example shown in FIG. 1, the expected fluid velocity M3 information is received from an external source. For example, the expected fluid velocity M3 may be predetermined information and as such may be provided by an external source and received by the processing unit 30. An example of an external source may be a memory unit having the expected fluid velocity M3 as predetermined information.

In the embodiment example shown in FIG. 2, the information about the expected fluid velocity M3 is determined by a determination unit 40 and provided to the processing unit 30.

In one embodiment, as shown, the expected fluid velocity M3 may be provided or received via an, optionally respective, interface.

The processing unit 30 is further adapted to generate a control output indicating a comparison result.

In a preferred embodiment, the control output may be sent to an output unit (not shown), such as a display, to generate a warning message if the comparison indicates that a discrepancy exists.

The processor unit 30 can preferably be configured to determine the downstream fluid velocity M2 using calibration parameters A, B corresponding to the predetermined water content fₛₒₗₗ. This can be done in particular using the equation described at the beginning.

Preferably, the processor unit 30 or the determination unit 40 is configured to determine the calibration parameters A, B and to provide them to the processor unit 30.

As shown in FIG. 2, the control system 100 may further comprise a determination unit 40. For example, and as shown, the determination unit 40 may be a part of the processing unit 30, preferably in that the processing unit 30 is configured to perform the functions of the determination unit 40. However, it may also be a physical component programmed to perform the functions of the determination unit 40.

The determination unit 40, when present as shown in FIG. 2, is configured to receive information about an upstream fluid velocity M4 upstream of the humidification unit 10. For example, the determination unit 40 may receive a measurement signal or information which indicates the fluid velocity M4 or from which the fluid velocity M4 is determinable.

The determination unit 40, when present as shown in FIG. 2, is further adapted to receive information about an upstream water content fᵢₙ of the breathing gas stream and to receive information about the predetermined water content fₛₒₗₗ.

The determination unit, if present as shown in FIG. 2, is further adapted to determine the expected fluid velocity M3 information from the upstream water content fᵢₙ, the predetermined water content fₛₒₗₗ, and the upstream fluid velocity M4, and to provide the expected fluid velocity M3 information to the processing unit 30.

Thus, the determination unit 40 may be configured to determine the information about the expected fluid velocity M3 independently of the humidification unit 10.

The control system 100 may include a further thermal anemometer 50, as shown in the embodiment shown in FIG. 2. The further thermal anemometer 50, when present as shown, is arranged upstream of the humidification unit 10 to determine the upstream fluid velocity M4 upstream of the humidification unit 10.

The thermal anemometer 20 and/or the further thermal anemometer 50 may be hot-wire anemometers in the illustrated embodiments.

In the illustrated embodiments, the thermal anemometer 20 and/or the further thermal anemometer 50 may be configured to be operated at a constant temperature.

The thermal anemometer 20 and/or the further thermal anemometer 50 may be formed as part of a Wheatstone bridge in the illustrated embodiments.

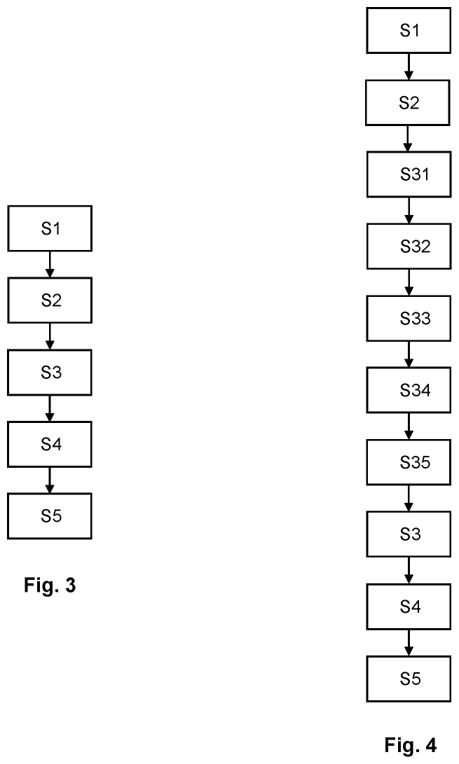

FIG. 3 shows an example of the process according to the invention.

Accordingly, the process according to the invention for generating a control output with a control system 100 according to the invention first has the step S1 in which the measurement signal M1 of the thermal anemometer 20 is received.

In step S2, the downstream fluid velocity M2 is determined from the measurement signal M1, as explained at the beginning.

In step S2, the determination of the downstream fluid velocity M2 can preferably be performed on the basis of the calibration parameters A, B as explained at the beginning, wherein the calibration parameters A, B correspond to the predetermined water content fₛₒₗₗ. The determination of the calibration parameters A, B can be done by calibration and the result of the calibration can be stored as predetermined information in a memory unit of the processor unit or in the determination unit.

In step S3, information about the expected fluid velocity M3 is received, for example, from a memory unit of the processor unit or from the determination unit as predetermined information or received from an external source.

In step S4, the comparison is made to see if there is a discrepancy between the downstream fluid velocity M2 and the expected fluid velocity M3.

In step S5, the control output indicating the comparison result is generated.

In a step not shown, the control output may be output for display or output for processing by a control or regulation unit of the humidification unit. Other types of processing of the control output are possible.

FIG. 4 shows a further embodiment of the process according to the invention.

This embodiment example corresponds to the process according to FIG. 2 with the difference that steps S31, S32, S33, S34 and S35 are additionally carried out between step S2 and step S3.

In step S31, information about the upstream fluid velocity M4 is received, which information may be provided, for example, as a measurement signal or as predetermined information.

For example, the upstream fluid velocity information M4 may be determined by means of another thermal anemometer 50, as already described for the control system 100.

In step S32, upstream water content information fᵢₙ is received as described earlier for control system 100.

In step S33, the information about the predetermined water content fₛₒₗₗ is received as already described for the control system 100.

In step S34, the expected fluid velocity M3 information is determined from the upstream water content fᵢₙ , the predetermined water content fₛₒₗₗ, and the upstream fluid velocity M4 as previously described for the control system 100.

In step S35, the information about the expected fluid velocity M3 is provided so that it can be received in step S3.

Since the process corresponds to the control system 100, an analogous, repetitive reproduction of the advantages, effects and possible embodiments is dispensed with. Accordingly, the advantages, effects and possible embodiments of the control system 100 are arbitrarily transferable to the process described herein.

All features described herein may be combined with each other in any manner, unless precluded or obviously incompatible.

While specific embodiments of the invention have been shown and described in detail to illustrate the application of the principles of the invention, it will be understood that the invention may be embodied otherwise without departing from such principles.

### LIST OF REFERENCE CHARACTERS

10 Humidification unit
20 Thermal anemometer, anemometer
30 Processor unit
40 Determination unit
50 Further thermal anemometer, further anemometer
100 Control system
fᵢₙ Upstream water content
fₛₒₗₗ Predetermined water content
M1 Measuring signal of the thermal anemometer
M2 Downstream fluid velocity
M3 Expected fluid velocity
M4 Upstream fluid velocity
S . . . steps of the process

What is claimed is:

1. A control system for a humidification unit, which is arranged to humidify a breathing gas stream in such a way that a predetermined water content is achieved, the control system comprising:
a thermal anemometer located downstream of the humidification unit; and
a processor unit configured to:
receive a measurement signal from the thermal anemometer and determine a downstream fluid velocity therefrom;
receive information about an expected fluid velocity corresponding to the predetermined water content;
perform a comparison to determine whether there is a discrepancy between the determined downstream fluid velocity and the expected fluid velocity; and
generate a control output that indicates a comparison result.

2. A control system according to claim 1, wherein the processor unit is configured to determine the downstream fluid velocity based on calibration parameters corresponding to the predetermined water content.

3. A control system according to claim 2, further comprising a determination unit configured to:
receive information about an upstream fluid velocity, upstream of the humidification unit;
receive information about an upstream water content of the breathing gas stream;
receive information about the predetermined water content;
determine the expected fluid velocity information from the predetermined water content, the upstream water content, and the upstream fluid velocity; and
provide the information about the expected fluid velocity for the processor unit.

4. A control system according to claim 3, further comprising a further thermal anemometer arranged upstream of the humidification unit to determine the upstream fluid velocity, upstream of the humidification unit.

5. A control system according to claim 4, wherein the thermal anemometer and/or the further thermal anemometer is a hot wire anemometer.

6. A control system according to claim 4, wherein the thermal anemometer and/or the further thermal anemometer is configured to be operated at a constant temperature.

7. A control system according to claim 4, wherein the thermal anemometer and/or the further thermal anemometer is formed as part of a Wheatstone bridge.

8. A control system according to claim 1, further comprising a determination unit configured to:
receive information about an upstream fluid velocity upstream of the humidification unit;
receive information about an upstream water content of the breathing gas stream;
receive information about the predetermined water content;
determine the expected fluid velocity information from the predetermined water content, the upstream water content, and the upstream fluid velocity; and
provide the information about the expected fluid velocity for the processor unit.

9. A control system according to claim 8, further comprising a further thermal anemometer arranged upstream of the humidification unit to determine the upstream fluid velocity upstream of the humidification unit.

10. A control system according to claim 1, wherein the thermal anemometer is a hot wire anemometer.

11. A control system according to claim 1, wherein the thermal anemometer is configured to be operated at a constant temperature.

12. A control system according to claim 1, wherein the thermal anemometer is formed as part of a Wheatstone bridge.

13. A process of generating a control output with a control system comprising a thermal anemometer, located downstream of a humidification unit, and a processor unit to generate a control output that indicates a comparison result, the process comprising the steps of:
with the processor unit, receiving the measurement signal of the thermal anemometer;
determining the downstream fluid velocity from the measurement signal;
receiving the information about an expected fluid velocity;
performing a comparison to determine whether there is a deviation between the downstream fluid velocity and the expected fluid velocity; and
generating the control output indicating the comparison result.

14. A process according to claim 13, wherein the downstream fluid velocity is determined based on the calibration parameters, corresponding to the predetermined water content.

15. A process of claim 13, further comprising the steps of:
receiving upstream fluid velocity information;
receiving information about the upstream water content of the breathing gas stream;
receiving the information about the predetermined water content;
determining the expected fluid velocity information from the upstream water content, the predetermined water content, and the upstream fluid velocity; and
providing expected fluid velocity information.
